(19) United States
(12) Patent Application Publication
(10) Pub. No.: US 2024/0173043 A1
(43) Pub. Date: May 30, 2024

(54) System and Method for Acoustic Treatment Using Targeted Placement of Low Dissolved Gas Liquids

(71) Applicant: Applaud Medical, Inc., San Francisco, CA (US)

(72) Inventors: William Behnke-Parks, San Francisco, CA (US); Yuri A. Pishchalnikov, Rohnert Park, CA (US); Paidamoyo J. Ewing, Burlingame, CA (US)

(21) Appl. No.: 18/520,704

(22) Filed: Nov. 28, 2023

Related U.S. Application Data
(60) Provisional application No. 63/385,057, filed on Nov. 28, 2022.

(51) Int. Cl. A61B 17/22

(52) U.S. Cl. CPC A61B 17/2202; A61B 2017/22008; A61B 2017/22009

(57) ABSTRACT

A method for treating a target biomineralization in a mammalian subject. The method includes placing a catheter in proximity to the target biomineralization; introducing a low-dissolved-gas liquid (LDGL) through the catheter, the LDGL having a dissolved-gas concentration of less than or equal to about 20% of the LDGL oxygen saturation level; flowing the LDGL towards the target biomineralization such that at least some of the LDGL is in a focal zone of one or more ultrasound transducers and between the target biomineralization and the ultrasound transducer(s), the focal zone aligned with the target biomineralization; applying focused ultrasound energy to the target biomineralization, the focused ultrasound energy passing through the LDGL; and forming cavitation bubbles, with the focused ultrasound energy, at a surface of the target biomineralization to fragment the target biomineralization. The LDGL reduces acoustic shielding in the focal zone compared to when the LDGL is not introduced.

### CROSS-REFERENCE TO RELATED APPLICATIONS

This application claims priority to U.S. Provisional Application No. 63/385,057, titled “System and Method for Acoustic Treatment Using Targeted Placement of LDGF Fluids,” filed on Nov. 28, 2022, which is hereby incorporated by reference.

### TECHNICAL FIELD

This invention relates generally to acoustic treatment of target regions while controlling or limiting gas bubble generation at or near said regions during treatment.

### BACKGROUND

While cavitation can be a useful mechanism for causing or enhancing acoustic treatment in biomedical applications including treatment of biomineralizations, cavitation can be a harmful mechanism for causing adverse bioeffects and dissipation of acoustic energy during the transmission along the acoustic path to the target. Some methods of treatment involve focused ultrasound from a geometrically shaped and/or from a phased array of acoustic elements or sources such as piezo-electric transducer sources. The acoustic source(s) form acoustic pressures which (during the negative-pressure phase) can exceed the spontaneous cavitation threshold pressure of the fluid in which the activity takes place, and thus one or more gas bubbles can be drawn from or nucleated in the focal zone.

Cavitation can be marked by the presence of such gas bubbles in a region of liquid, sometimes forming a gas bubble “cloud” situated along the acoustic path between the acoustic source and the intended focal or treatment zone. The bubble formation along the acoustic path is undesired because gas bubbles can “shield” the intended target region due to the dramatic difference in acoustic impedance between the gas (bubbles) and the surrounding liquid propagation medium. In such instances the acoustic energy can be reflected, scattered or absorbed by the gas bubbles instead of being delivered in the intended target or focal zone. This dissipation of acoustic energy thus limits the effectiveness with which ultrasound therapy and similar treatments can be delivered. Acoustic treatment methods and systems rely on the propagation capability of the subject tissue and intervening tissues and fluid to transmit the acoustic energy to the target. These tissues and fluids, in some examples blood or urine, carry considerable concentrations of dissolved gasses which during an acoustic treatment can form manifest in gas bubbles or bubble clouds, as described, which are counter-productive in many situations.

### SUMMARY

Example embodiments described herein have innovative features, no single one of which is indispensable or solely responsible for their desirable attributes. The following description and drawings set forth certain illustrative implementations of the disclosure in detail, which are indicative of several exemplary ways in which the various principles of the disclosure may be carried out. The illustrative examples, however, are not exhaustive of the many possible embodiments of the disclosure. Without limiting the scope of the claims, some of the advantageous features will now be summarized. Other objects, advantages, and novel features of the disclosure will be set forth in the following detailed description of the disclosure when considered in conjunction with the drawings, which are intended to illustrate, not limit, the invention.

An aspect of the invention is directed to a method for treating a target biomineralization in a mammalian subject. The method comprises placing a catheter in proximity to the target biomineralization, the catheter fluidly coupled to a source of a low-dissolved-gas liquid (LDGL), the LDGL having a dissolved-gas concentration of less than or equal to about 20% of an oxygen saturation level of the LDGL; introducing the LDGL through the catheter; flowing the LDGL towards the target biomineralization such that at least some of the LDGL is in a focal zone of one or more ultrasound transducers and between the target biomineralization and the one or more ultrasound transducers, the focal zone aligned with the target biomineralization; applying focused ultrasound energy to the target biomineralization with the one or more ultrasound transducers, the focused ultrasound energy passing through the LDGL; and forming cavitation bubbles, with the focused ultrasound energy, at a surface of the target biomineralization to fragment the target biomineralization. The LDGL reduces acoustic shielding in the focal zone between the target biomineralization and the one or more ultrasound transducers compared to when the LDGL is not introduced.

In one or more embodiments, the catheter is placed upstream of the target biomineralization such that bodily fluids flow from the catheter towards the target biomineralization. In one or more embodiments, the LDGL reduces the acoustic shielding in a region that begins at least 2 mm from the surface of the target biomineralization. In one or more embodiments, the LDGL reduces spontaneous cavitation in the region compared to when the LDGL is not introduced.

In one or more embodiments, the method further comprises introducing a stream of the LDGL through the catheter. In one or more embodiments, when the at least some of the LDGL is in the ultrasound focal zone and between the target biomineralization and the one or more ultrasound transducers, a boundary layer of bodily fluids is located along the surface of the target biomineralization, and the cavitation bubbles are formed in the boundary layer of bodily fluids. In one or more embodiments, a dissolved-oxygen concentration of the LDGL less than or equal to about 2 mg/L.

Another aspect of the invention is directed to a method for treating a target biomineralization in a mammalian subject. The method comprises placing a catheter in proximity to the target biomineralization, the catheter fluidly coupled to a source of a low-dissolved-gas liquid (LDGL) and to a source of microbubbles, the LDGL having a dissolved-gas concentration of less than or equal to about 20% of an oxygen saturation level of the LDGL; introducing the microbubbles through the catheter; flowing the microbubbles towards the target biomineralization such that at least some of the microbubbles accumulate on a surface of the target biomineralization, the at least some of the microbubbles in a focal zone of one or more ultrasound transducers and between the target biomineralization and the one or more ultrasound transducers, the focal zone aligned with the target biomineralization; introducing the LDGL through the catheter; flowing the LDGL towards the target biomineralization such that at least some of the LDGL is in the focal zone of the one or more ultrasound transducers and between the target biomineralization and the one or more ultrasound transducers; applying focused ultrasound energy to the target biomineralization with the one or more ultrasound transducers, the focused ultrasound energy passing through the LDGL; and forming cavitation bubbles with the accumulated microbubbles on the surface of the target biomineralization to fragment the target biomineralization, the cavitation bubbles formed with the focused ultrasound energy. The LDGL reduces an acoustic shielding in the focal zone between the target biomineralization and the one or more ultrasound transducers compared to when the LDGL is not introduced.

In one or more embodiments, the catheter is placed upstream of the target biomineralization such that bodily fluids flow from the catheter towards the target biomineralization. In one or more embodiments, the LDGL reduces the acoustic shielding in a region that begins at least 2 mm from the surface of the target biomineralization. In one or more embodiments, reduces spontaneous cavitation in the region compared to when the LDGL is not introduced.

In one or more embodiments, the method further comprises introducing a stream of the LDGL through the catheter. In one or more embodiments, when the at least some of the LDGL is in the focal zone and between the target biomineralization and the one or more ultrasound transducers, a boundary layer of bodily fluids is located along the surface of the target biomineralization, and the accumulated microbubbles are in the boundary layer of bodily fluids. In one or more embodiments, a Y-coupling fluidly couples the catheter to the source of the LDGL and to the source of microbubbles.

Another aspect of the invention is directed to a method for treating a target biomineralization in a mammalian subject. The method comprises placing a first catheter in proximity to the target biomineralization, the first catheter fluidly coupled to a source of microbubbles; introducing the microbubbles through the first catheter; flowing the microbubbles towards the target biomineralization such that at least some of the microbubbles accumulate on a surface of the target biomineralization, the at least some of the microbubbles in a focal zone of one or more ultrasound transducers and between the target biomineralization and the one or more ultrasound transducers, the focal zone aligned with the target biomineralization; placing a second catheter in proximity to the target biomineralization, the second catheter fluidly coupled to a source of a low-dissolved-gas liquid (LDGL), the LDGL having a dissolved-gas concentration of less than or equal to about 20% of an oxygen saturation level of the LDGL; introducing the LDGL through the second catheter; flowing the LDGL towards the target biomineralization such that at least some of the LDGL is in the focal zone of the one or more ultrasound transducers and between the target biomineralization and the one or more ultrasound transducers; applying focused ultrasound energy to the target biomineralization with the one or more ultrasound transducers, the focused ultrasound energy passing through the LDGL; and forming cavitation bubbles with the accumulated microbubbles on the surface of the target biomineralization to fragment the target biomineralization, the cavitation bubbles formed with the focused ultrasound energy. The LDGL reduces an acoustic shielding in the focal zone between the target biomineralization and the one or more ultrasound transducers compared to when the LDGL is not introduced.

In one or more embodiments, the first and/or second catheters are placed upstream of the target biomineralization such that bodily fluids flow from the first and second catheters towards the target biomineralization. In one or more embodiments, the LDGL reduces the acoustic shielding in a region that begins at least 2 mm from the surface of the target biomineralization. In one or more embodiments, the LDGL reduces spontaneous cavitation in the region compared to when the LDGL is not introduced.

In one or more embodiments, the method further comprises introducing a stream of the LDGL through the catheter. In one or more embodiments, when the at least some of the LDGL is in the focal zone and between the target biomineralization and the one or more ultrasound transducers, a boundary layer of bodily fluids is located along the surface of the target biomineralization, and the accumulated microbubbles are in the boundary layer of bodily fluids.

### BRIEF DESCRIPTION OF THE DRAWINGS

For a fuller understanding of the nature and advantages of the concepts disclosed herein, reference is made to the detailed description of preferred embodiments and the accompanying drawings.

FIG. 1 is an example graph that illustrates the behavior of a fluidic environment during ultrasound treatment according to an embodiment.

FIG. 2 is a flow chart of a method for treating a target biomineralization in a mammalian subject according to an embodiment.

FIG. 3 is a simplified view of an anatomical environment to illustrate steps 201 and 202 according to an embodiment.

FIG. 4 is a simplified view of an anatomical environment to illustrate step 203 according to an embodiment.

FIG. 5 is a simplified view of an anatomical environment to illustrate step 204 according to an embodiment.

FIG. 6 is an enlarged view of a region in FIG. 5 to illustrate step 205 according to an embodiment.

FIG. 7 is a flow chart of a method for treating a target biomineralization in a mammalian subject according to another embodiment.

FIG. 8A is a simplified view of an anatomical environment to illustrate steps 701 and 702 of FIG. 7 according to a first embodiment.

FIG. 8B is a simplified view of an anatomical environment to illustrate steps 701 and 702 of FIG. 7 according to a second embodiment.

FIG. 9A is a simplified view of an anatomical environment to illustrate step 703 of FIG. 7 according to the first embodiment.

FIG. 9B is a simplified view of an anatomical environment to illustrate step 703 of FIG. 7 according to the second embodiment.

FIG. 10A is a simplified view of an anatomical environment to illustrate step 704 of FIG. 7 according to the first embodiment.

FIG. 10B is a simplified view of an anatomical environment to illustrate step 704 of FIG. 7 according to the second embodiment.

FIG. 11A is a simplified view of an anatomical environment to illustrate step 705 of FIG. 7 according to the first embodiment.

FIG. 11B is a simplified view of an anatomical environment to illustrate step 705 of FIG. 7 according to the second embodiment.

FIG. 12A is a simplified view of an anatomical environment to illustrate step 706 of FIG. 7 according to the first embodiment.

FIG. 12B is a simplified view of an anatomical environment to illustrate step 706 of FIG. 7 according to the second embodiment.

FIG. 13 is an enlarged view of region in FIGS. 12A and 12B to illustrate step 707 of FIG. 7 according to the first and second embodiments.

FIG. 14 is a block diagram of an apparatus for manufacturing an LDGL according to an embodiment.

FIGS. 15 and 16 are block diagrams of example LDGL storage containers.

### DETAILED DESCRIPTION

A low dissolved-gas liquid (LDGL) is introduced in proximity to a target biomineralization. The LDGL has a lower dissolved-gas concentration compared to the equivalent unprocessed liquid and/or compared to the bodily fluids where it is introduced. The dissolved-gas concentration can be measured with a dissolved oxygen meter. The amount of dissolved oxygen is proportional to all the amount of all dissolved gases in a liquid such as water in normal conditions and measurement of the dissolved oxygen level is the most common method of determining the amount of gas dissolved in a liquid. The dissolved oxygen meters typically show the level of dissolved oxygen in absolute units of mg/L, ppm, or as a percentage relative to the oxygen saturation level of the liquid. The dissolved-gas concentration (e.g., dissolved oxygen concentration) can be measured at normal temperature and pressure (NTP) (i.e., 20° C. and 1 ATM). The LDGL can have a dissolved-gas concentration of less than or equal to about 30% of dissolved gas saturation level (e.g., oxygen saturation level) of the LDGL, less than or equal to about 20% (e.g., about 1% to about 20%) of dissolved gas saturation level (e.g., oxygen saturation level) of the LDGL, less than or equal to about 10% (e.g., about 1% to about 10%) of dissolved gas saturation level (e.g., oxygen saturation level) of the LDGL and/or less than or equal to about 5% (e.g., about 1% to about 5%) of dissolved gas saturation level (e.g., oxygen saturation level) of the LDGL. As used herein, “about” means plus or minus 10% of the corresponding value. The LDGL can be or can comprise water, saline, saline, and/or another liquid. The LDGL can be biocompatible and suitable to introduce into a mammalian subject, such as a human. For example, the LDGL can be or can comprise a sterilized liquid, such as sterile water for injection.

The LDGL is introduced or flows into a focal zone of an ultrasound transducer. Focused ultrasound energy is applied to the target biomineralization within the focal zone. The LDGL reduces acoustic shielding and/or spontaneous cavitation in a portion of the focal zone that starts at least 2 mm from the surface of the target biomineralization at the applied acoustic pressures. Cavitation is promoted in the bodily fluids that remain in a boundary layer at the surface of the target biomineralization. The cavitation (e.g., cavitation bubbles) in the bodily fluids can fragment the target biomineralization to allow the target biomineralization to pass through the mammalian subject. The bodily fluids can have a dissolved gas concentration of about 40% to about 60% of dissolved gas saturation level. The LDGL has a lower dissolved-gas concentration (e.g., 2 mg/L of dissolved oxygen content or 20% of dissolved gas saturation level) than the bodily fluids where it is introduced.

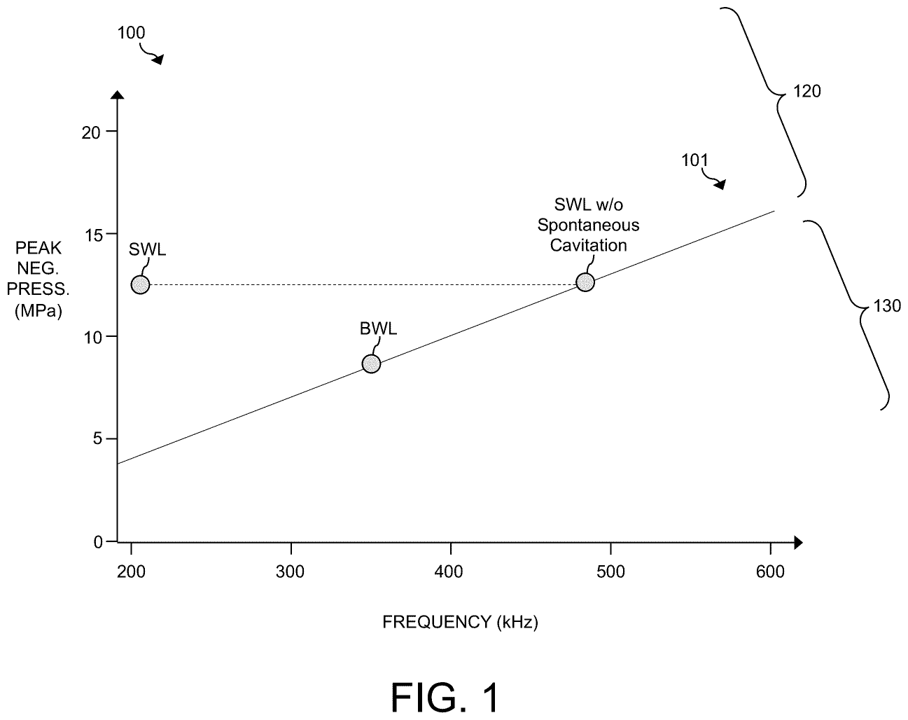

FIG. 1 is an example graph 100 that illustrates the behavior of a fluidic environment during ultrasound treatment where the vertical axis depicts peak negative acoustic pressure (MPa) and the horizontal axis depicts the center frequency (kHz) of the acoustic energy packet(s) delivered to the target. A line 101 separates two operating regimes. Above line 101 is a high-energy regime 120 where high-energy cavitation occurs. Below line 101 is a low-energy regime 130 that is dominated by acoustic shielding. Cavitation generally does not occur in the low-energy regime 130. The high-energy regime 120 generally corresponds to higher peak negative pressures and/or lower frequencies compared to the low-energy regime 130. The position of the line 101 is dependent on the gas concentration of the fluid. A higher gas concentration of the fluid causes the line 101 to move downward; a lower gas concentration of the fluid causes the line 101 to move upward.

Though operating in the high-energy regime 120 is desirable for certain treatments, such as burst-wave lithotripsy (BWL) and shock-wave lithotripsy (SWL), cavitation away from the surface of the target biomineralization can cause undesirable bioeffects, such as hematoma, especially to soft tissues. Typical SWL devices operate in the 10-12 MPa range. BWL devices operate at lower peak negative pressures, such as in the 8 MPa range.

As can be seen, in the case of SWL, to reduce spontaneous cavitation (and unwanted bubble cloud shielding) the amplitude of the acoustic energy used for treatment would have to be reduced to a level that is not effective or is less effective than desired. In this example we see that only at about 500 kHz center frequency treatment would we have sufficient head room in the phase diagram shown to reduce spontaneous cavitation, and this traditionally would be undesirably weak (amplitude/pressure/power) to achieve stone comminution effectively.

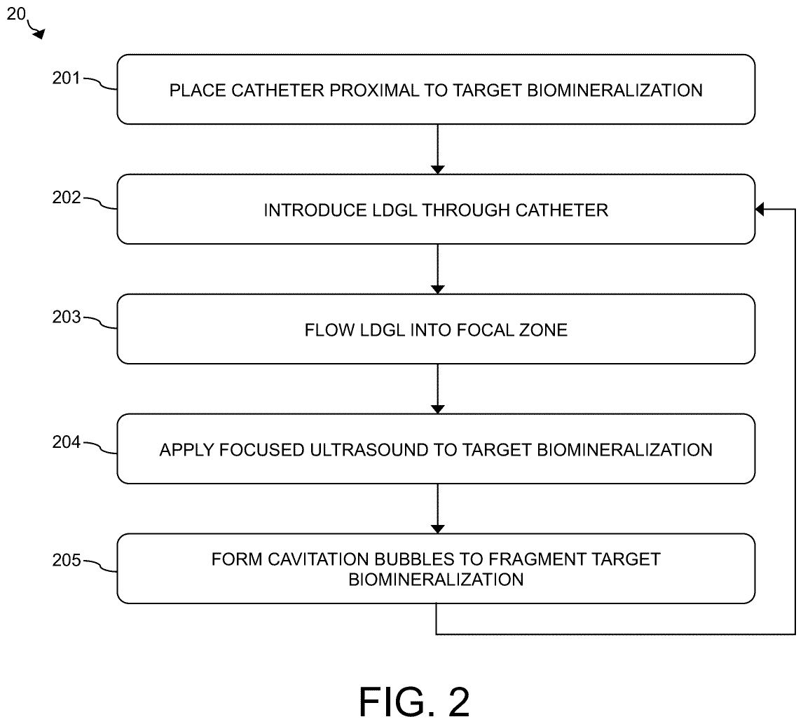

FIG. 2 is a flow chart of a method 20 for treating a target biomineralization in a mammalian subject according to an embodiment.

In step 201, a catheter is placed in a target location that is near or in proximity to the target biomineralization. The catheter can be placed using imaging feedback, such as ultrasound diagnostic imaging, magnetic resonance imaging (MRI), and/or visual/optical imaging. The catheter can be placed upstream of the target biomineralization where the bodily fluid(s) flow around the catheter and towards the target biomineralization. The catheter is fluidly coupled to a source of LDGL. In an alternative embodiment, the catheter can be placed downstream of the target biomineralization where the bodily fluid(s) flow around the catheter and away from the target biomineralization.

In step 202, an LDGL is introduced through the catheter at the target location. The LDGL can be introduced as a stream or as a single volume (e.g., a slug), which can have a predetermined volume.

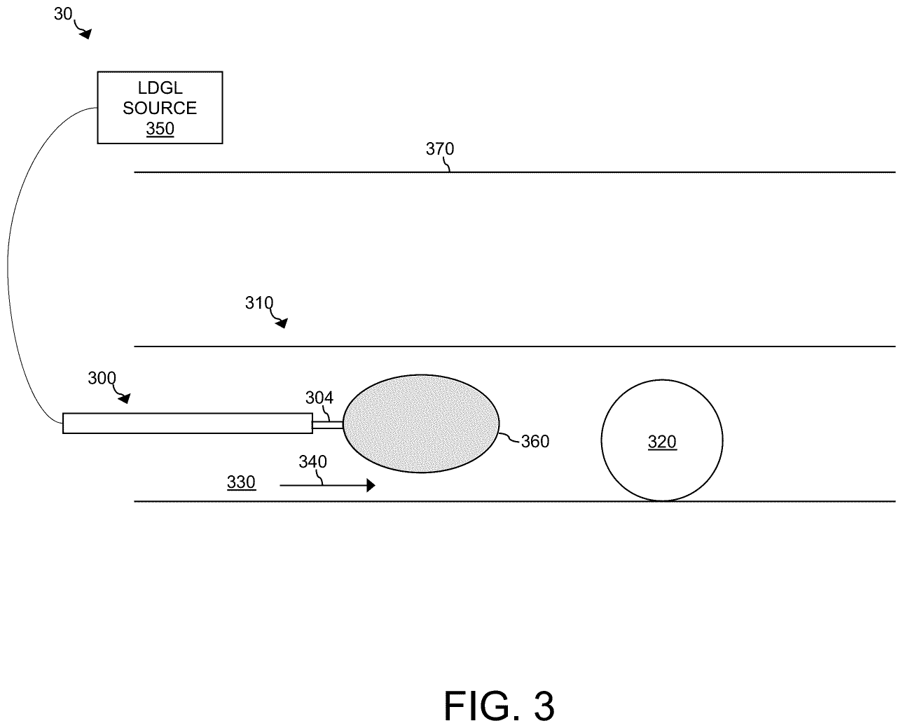

FIG. 3 is a simplified view of an anatomical environment 30 to illustrate steps 201 and 202 according to an embodiment. In environment 30, a catheter 300 is placed in a body vessel 310, such as a renal calyx, upstream of a target kidney stone 320. Bodily fluids 330, such as urine, flows in direction 340 from the catheter 300 towards the body vessel 310. The catheter 300 can be fluidly coupled (e.g., via a tube) to an LDGL source 350. The LDGL source 350 can be or include a container, a reservoir, a vessel, and/or another source. The catheter 300 includes a tip or delivery end 304 through which LDGL 360 is introduced. The skin surface 370 is illustrated for reference.

In step 203, the LDGL flows towards the target biomineralization and into the focal zone of the therapeutic focused ultrasound device. The LDGL can partially or fully displace the bodily fluids in the focal zone. A boundary layer of bodily fluids can remain at and/or along the surface of the target biomineralization.

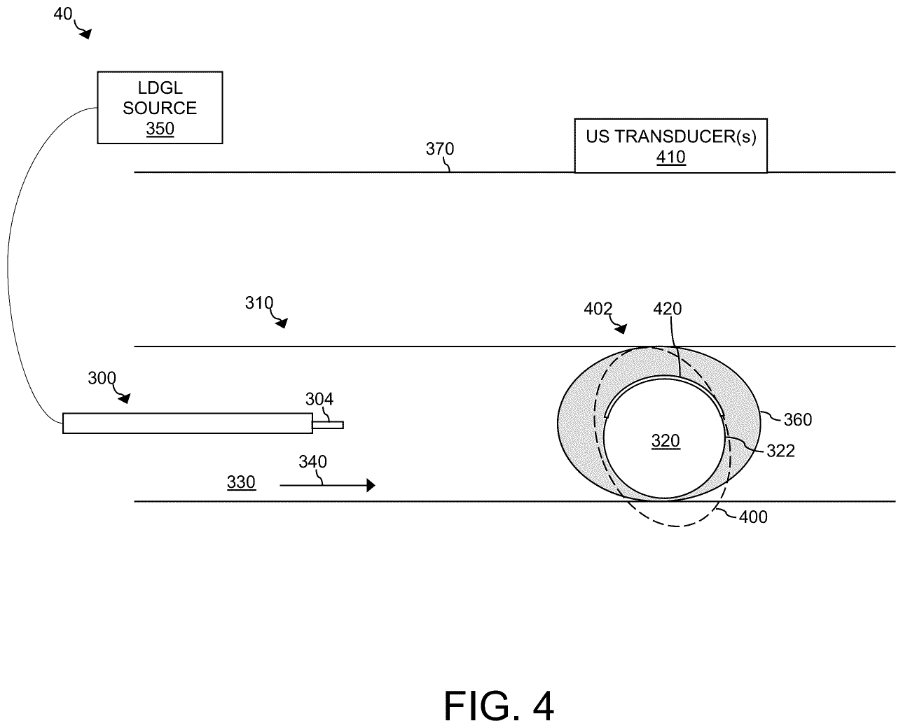

FIG. 4 is a simplified view of an anatomical environment 40 to illustrate step 203 according to an embodiment. Anatomical environments 30 and 40 are the same except that in anatomical environment 40 the LDGL 360 has flowed in direction 340 toward the target kidney stone 320 and into the focal zone 400 of one or more therapeutic ultrasound transducers 410. The LDGL 360 has displaced some or all of the bodily fluids 300 in the focal zone 400, including the portion 402 of the focal zone 400 between the target kidney stone 320 and the therapeutic ultrasound transducer(s) 410, to thereby reduce the dissolved-gas concentration in the focal zone 400.

A thin layer such as a boundary layer 420 of the bodily fluids 300 can remain along some or all of the surface 322 of the target kidney stone 320 such as in the portion 402 of the focal zone 400. Cavitation starts to occur at the surface of the target kidney stone 320 before the surrounding liquid due to increased acoustic pressure at the acoustically rigid surface of kidney stones. This increase of the acoustic pressure at the surface of kidney stones is caused by a mismatch of acoustic impedances of the biomineralization and the surrounding liquid.

In step 204, focused ultrasound energy is applied to the target biomineralization using one or more ultrasound transducers. The ultrasound energy can be focused using a geometric focus (e.g., a bowl reflector as in high-intensity focused ultrasound (HIFU) and/or an electronic focus (e.g., a phased ultrasound array). The focused ultrasound energy passes through the LDGL to reach the target biomineralization. Some or all of the LDGL is between the one or more ultrasound transducers and the target biomineralization.

Since spontaneous cavitation in the LDGL occurs at higher pressures, the focused ultrasound can be applied at a higher peak negative pressure and/or at a lower frequency compared to when the LDGL is not introduced.

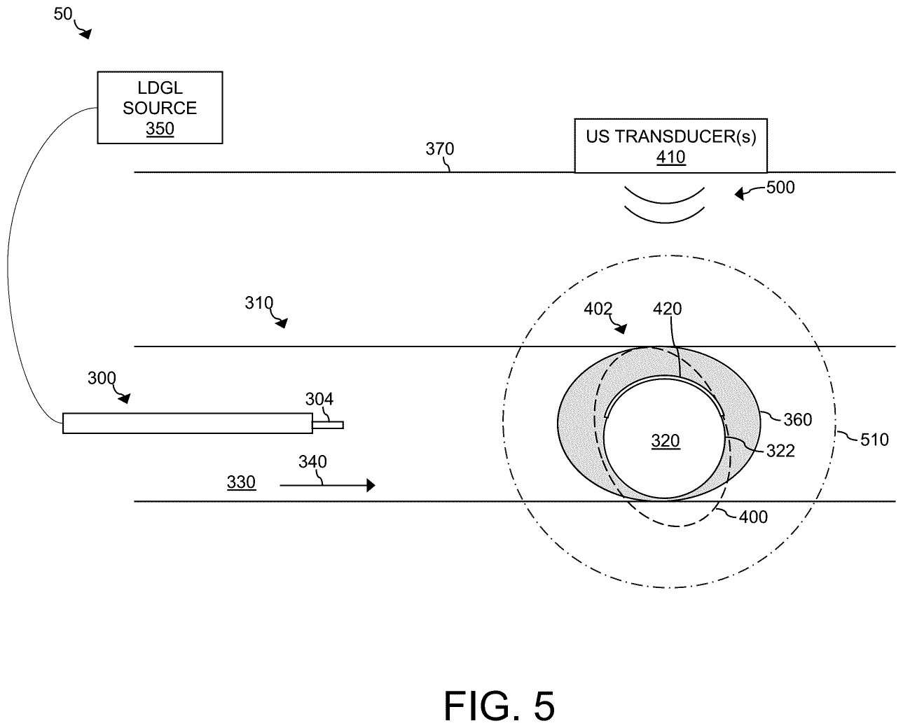

FIG. 5 is a simplified view of an anatomical environment 50 to illustrate step 204 according to an embodiment. Anatomical environments 40 and 50 are the same except that in anatomical environment 50, the therapeutic ultrasound transducer(s) 410 produces focused ultrasound energy 500 that forms and/or defines the focal zone 400.

In step 205, the focused ultrasound energy forms cavitation bubbles that fragment the target biomineralization. The cavitation bubbles can be formed in the boundary layer along the external surface of the target biomineralization such as in the portion of the focal zone between the target biomineralization and the one or more ultrasound transducers. Gas-bubble shielding can be reduced in the LDGL such that the focused ultrasound energy can pass through the LDGL with reduced and/or minimal reflection, scatter, and/or absorption by gas bubbles. These gas bubbles are formed when the focused ultrasound energy passes through a bodily fluid but are not present (or have a lower concentration) in the LDGL. Additionally or alternatively, cavitation bubble formation can be reduced in the LDGL to reduce the undesirable bioeffects of high-energy cavitation to the surrounding anatomy.

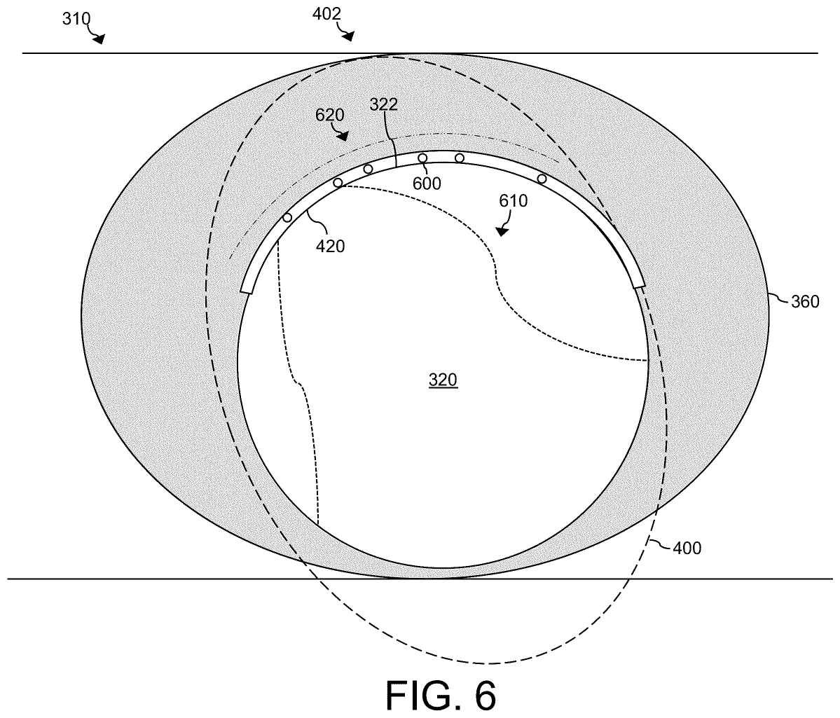

FIG. 6 is an enlarged view of region 510 in FIG. 5 to illustrate step 205 according to an embodiment. The focused ultrasound energy produces cavitation bubbles 600 in the boundary layer 420 of bodily fluids along the target kidney stone 320 in the portion 402 of the focal zone 400 between the target kidney stone 320 and the therapeutic ultrasound transducer(s) 410. As noted, the boundary layer 420 can extend along additional surfaces of the target kidney stone 320. When the cavitation bubbles 600 collapse (e.g., with positive acoustic pressures), energy is released to fragment 610 the target kidney stone 320.

The LDGL reduces gas-bubble shielding and/or spontaneous cavitation bubble formation in a region 620 that begins at least 2 mm from the surface 322 of the target kidney stone 320. The region 620 can begin at 2 mm from the surface 322, at 3 mm from the surface 322, at 4 mm from the surface, and any other value or range between any two of the foregoing values. The region 620 is within the portion 402 of the focal zone 400.

In some embodiments, steps 202-205 can be repeated in multiple treatment cycles. The treatment cycles can be repeated immediately or after a delay period.

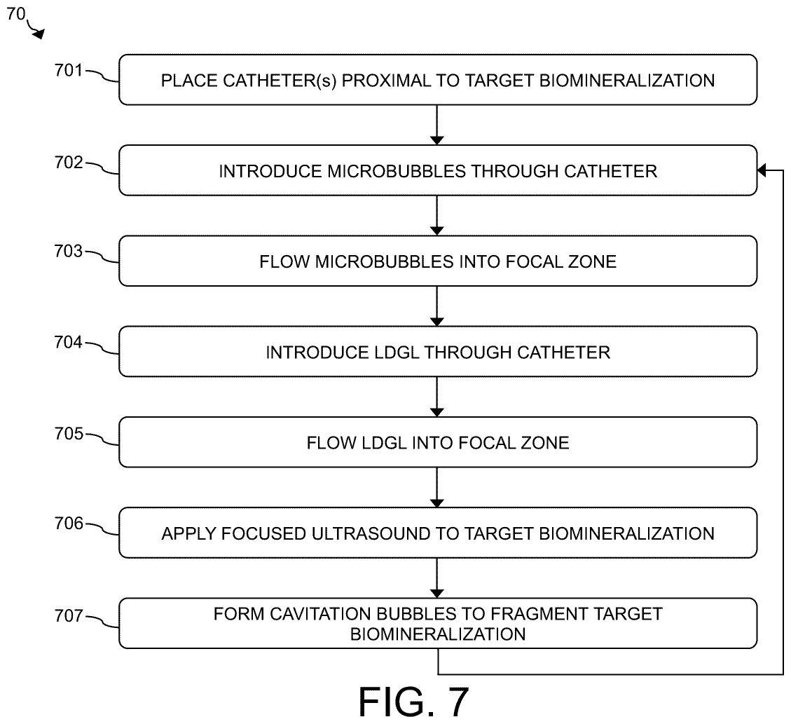

FIG. 7 is a flow chart of a method 70 for treating a target biomineralization in a mammalian subject according to another embodiment.

In step 701, one or more catheters is/are placed in a target location that is near or in proximity to the target biomineralization. The catheter(s) can be placed using imaging feedback, such as ultrasound diagnostic imaging, magnetic resonance imaging (MRI), and/or visual/optical imaging. The catheter(s) can be placed upstream of the target biomineralization where the bodily fluid(s) flow around the catheter and towards the target biomineralization. In an alternative embodiment, the catheter(s) can be placed downstream of the target biomineralization where the bodily fluid(s) flow around the catheter and away from the target biomineralization. In an example, one catheter is placed upstream of the target biomineralization and the other catheter is placed downstream of the target biomineralization.

In an embodiment, one catheter is placed in the target location. The catheter is fluidly coupled, such as through a Y-connector, to a source of LDGL and to a source of microbubbles. In another embodiment, two catheters are placed in the target location. A first catheter is fluidly coupled to a source of LDGL. A second catheter is fluidly coupled to a source of microbubbles.

In step 702, microbubbles are introduced through the catheter at the target location. The microbubbles can be the same as disclosed in U.S. Patent Publication No. 2021/0015511, titled “System And Method For Communition Of Biomineralization Using Microbubbles,” published on Jan. 21, 2021 and/or U.S. Patent Publication No. 2022/0000509, titled “Ultrasound Device for Use with Synthetic Cavitation Nuclei,” published on Jan. 6, 2022, which are hereby incorporated by reference. The microbubbles can be introduced as a stream or as a single volume (e.g., a slug), which can have a predetermined volume.

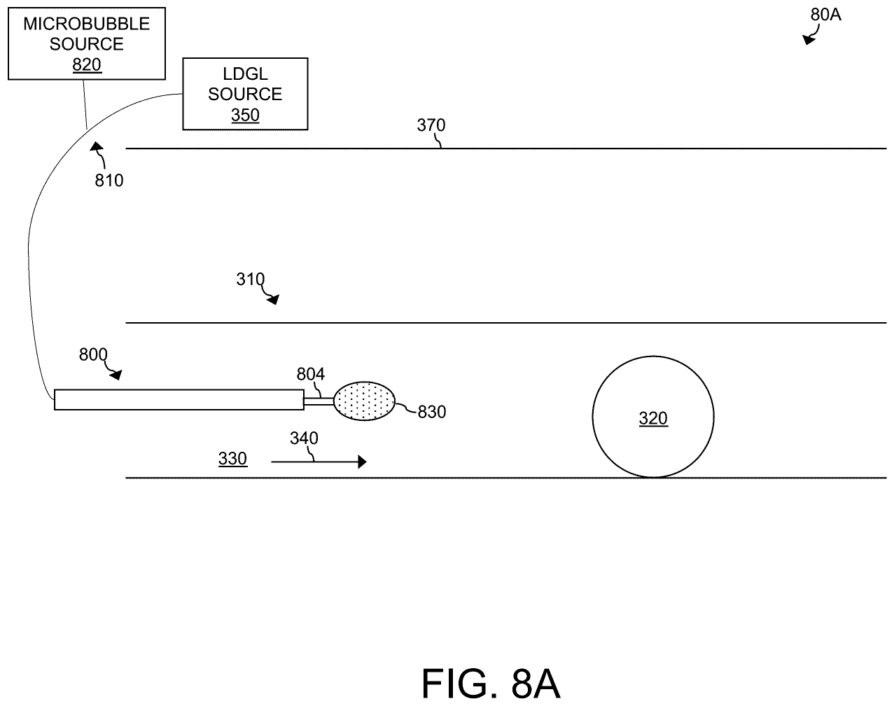

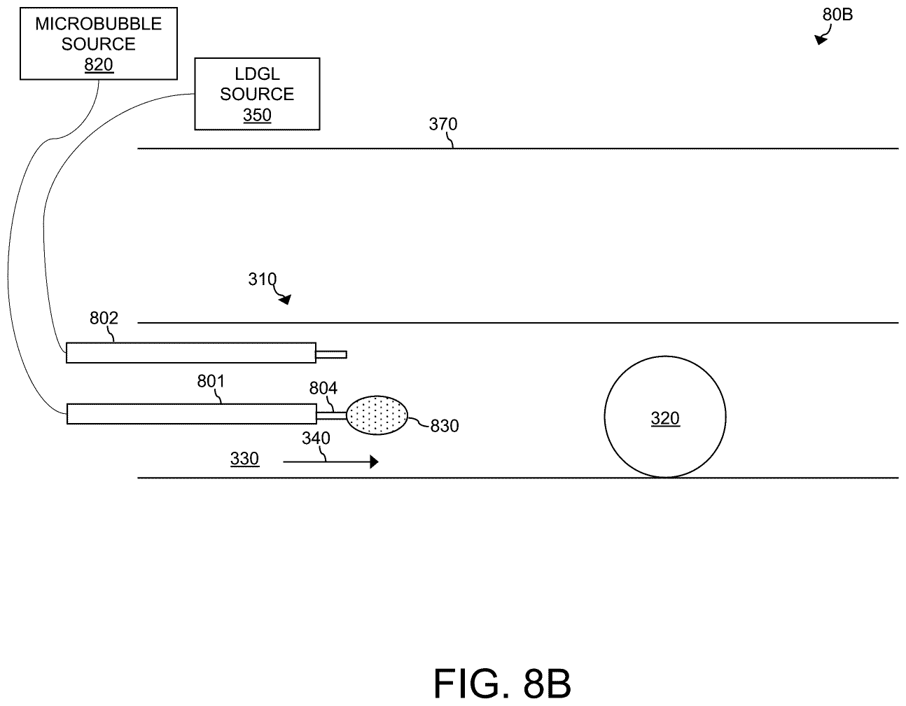

FIG. 8A is a simplified view of an anatomical environment 80A to illustrate steps 701 and 702 according to a first embodiment. Environment 80A is the same as environment 30 except that in environment 80A, a Y-connector 810 fluidly couples the catheter 800 placed in the body vessel 310 to a microbubble source 820 and to the LDGL source 350. The Y-connector 810 can include a valve that allow the catheter 800 to be fluidly coupled to the microbubble source 820 when the valve is in a first position or state and to the LDGL source 350 when the valve is in a second position or state. The microbubble source 820 can be or include a container, a reservoir, a vessel, and/or another source. A volume of microbubbles 830 is introduced through a tip or delivery end 804 of the catheter 800.

FIG. 8B is a simplified view of an anatomical environment 80B to illustrate steps 701 and 702 according to a second embodiment. Environment 80B is the same as environment 80A except that in environment 80B, two catheters 801, 802 are placed in the body vessel 310 upstream of the target kidney stone 320. The first catheter 801 is fluidly coupled to the microbubble source 820. The second catheter 802 is fluidly coupled to the LDGL source 350. The volume of microbubbles 830 is introduced through the tip or delivery end 804 of the first catheter 801.

In step 703, the microbubbles flow towards the target biomineralization and into the focal zone of the therapeutic focused ultrasound device. At least some of the microbubbles accumulate on and/or along the surface of the target biomineralization.

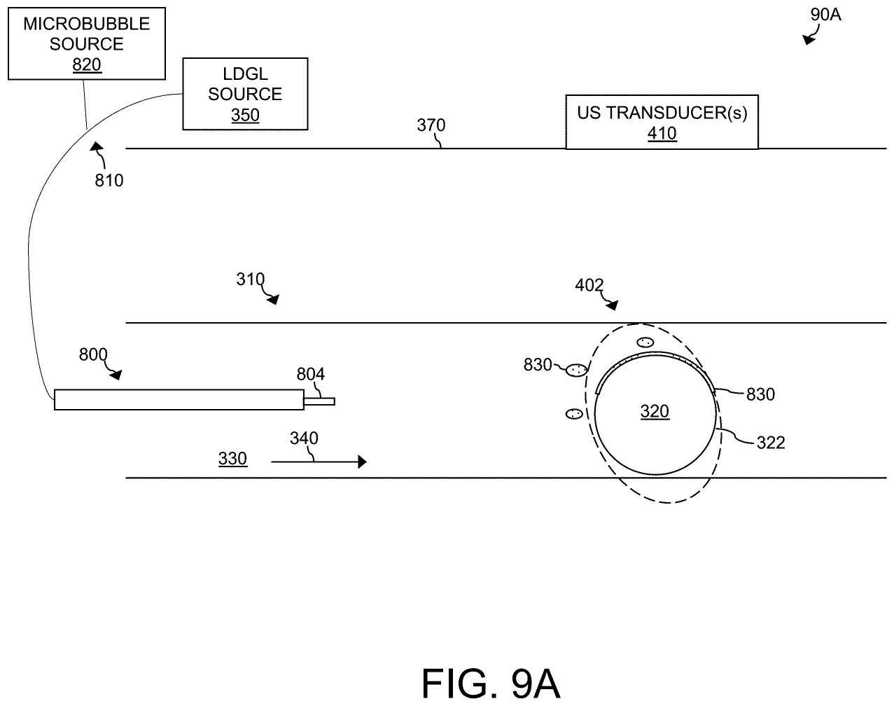

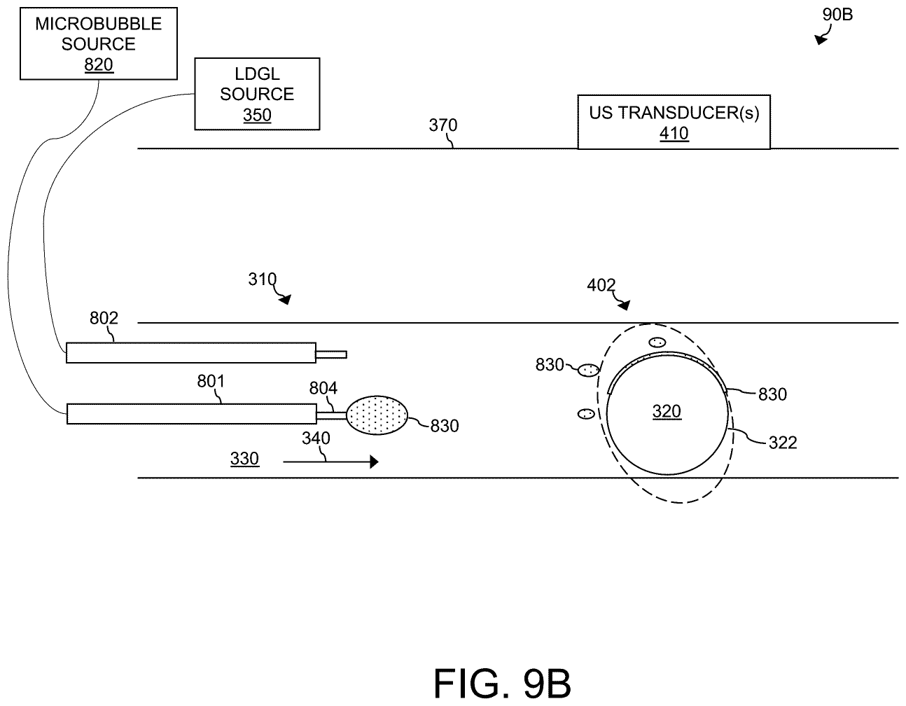

FIG. 9A is a simplified view of an anatomical environment 90A to illustrate step 703 according to the first embodiment. Anatomical environments 80A and 90A are the same except that in anatomical environment 90A the microbubbles 830 have flowed in direction 340 toward the target kidney stone 320 and into the focal zone 400 of a therapeutic ultrasound transducer(s) 410. At least some of the microbubbles 830 have accumulated on and/or along the surface 322 of the target kidney stone 320 including in the portion 402 of the focal zone 400 between the target kidney stone 320 and the therapeutic ultrasound transducer(s) 410.

FIG. 9B is a simplified view of an anatomical environment 90B to illustrate step 703 according to the second embodiment. With respect to step 703, anatomical environments 90A and 90B are the same.

In step 704, an LDGL is introduced through a catheter at the target location. The same catheter can be used to introduce the microbubbles and the LDGL. Alternatively, different catheters can be used to introduce the microbubbles and the LDGL. Alternatively, a multi-lumen catheter can be used to introduce the microbubbles and the LDGL. The LDGL can be introduced as a stream or as a single volume (e.g., a slug), which can have a predetermined volume.

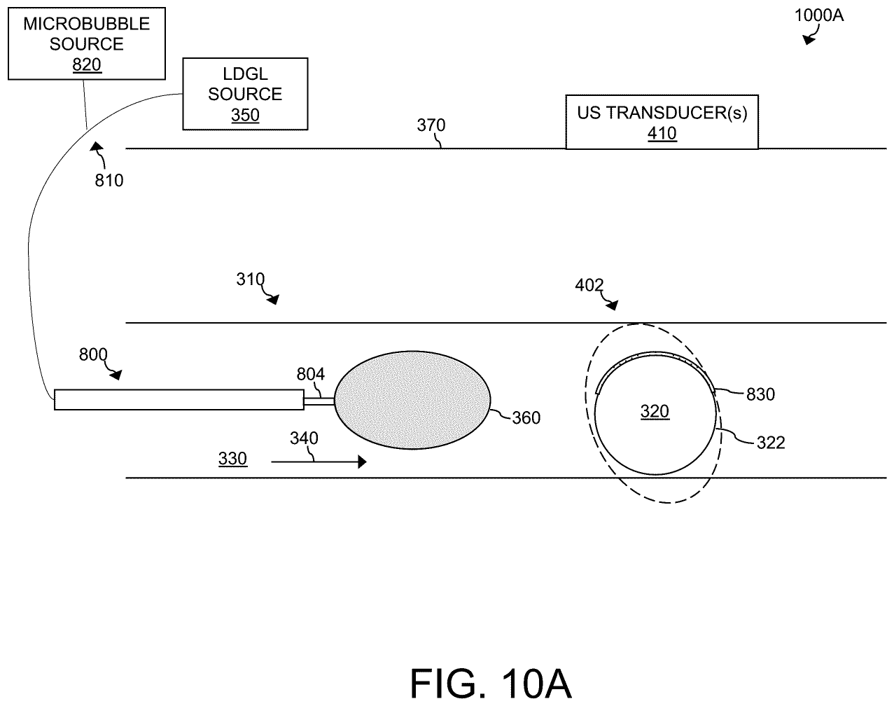

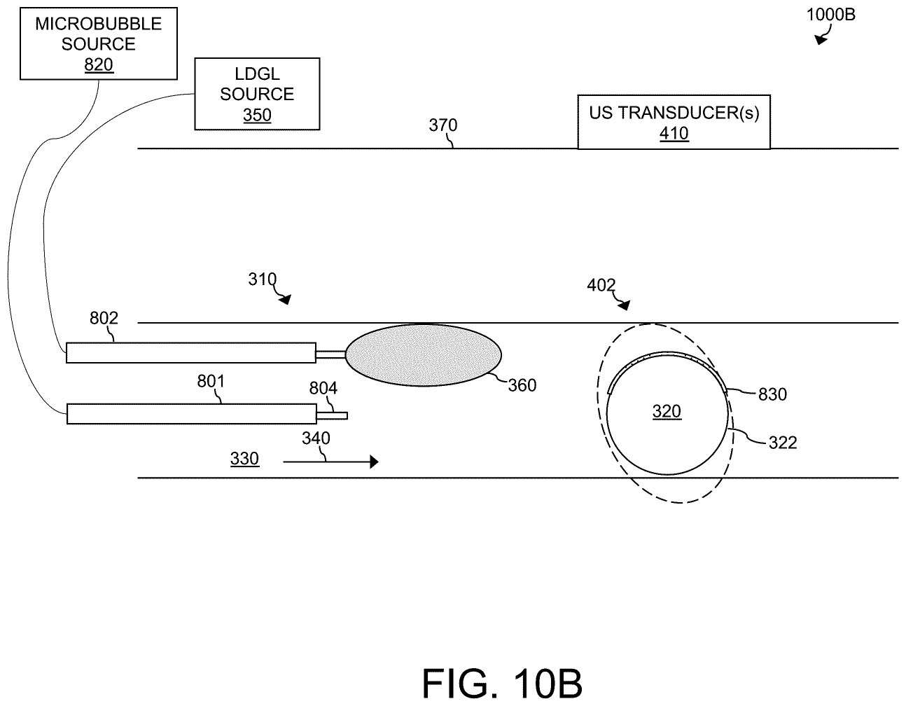

FIG. 10A is a simplified view of an anatomical environment 1000A to illustrate step 704 according to the first embodiment. In environment 1000A, the LDGL 360 is introduced through the catheter 800. When the Y-connector 810 includes a valve, the valve can be in the second position or state to fluidly couple the catheter 800 to the LDGL source 350.

FIG. 10B is a simplified view of an anatomical environment 1000B to illustrate step 704 according to the second embodiment. In environment 1000B, the LDGL 360 is introduced through the catheter 800. When the Y-connector 810 includes a valve, the valve can be in the second position or state to fluidly couple the catheter 800 to the LDGL source 350.

In step 705, the LDGL flows towards the target biomineralization and into the focal zone of the therapeutic focused ultrasound device. The LDGL can partially or fully displace the bodily fluids and any microbubbles, in the focal zone, that are not accumulated at/along the surface of the target biomineralization. The accumulated microbubbles remain at and/or along the surface of the target biomineralization. The accumulated microbubbles can be in a boundary layer of bodily fluids along the surface of the target biomineralization.

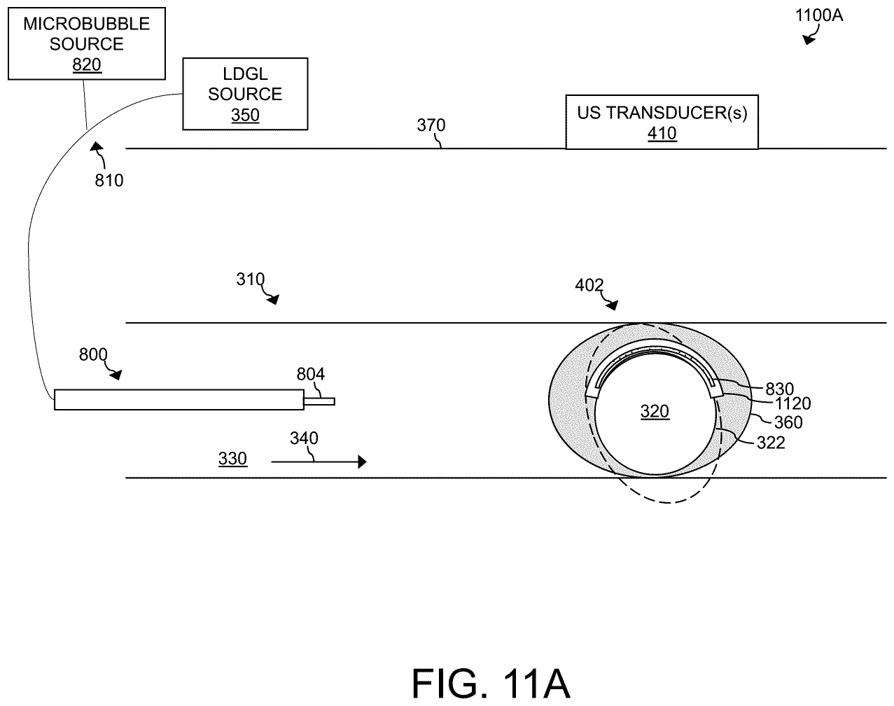

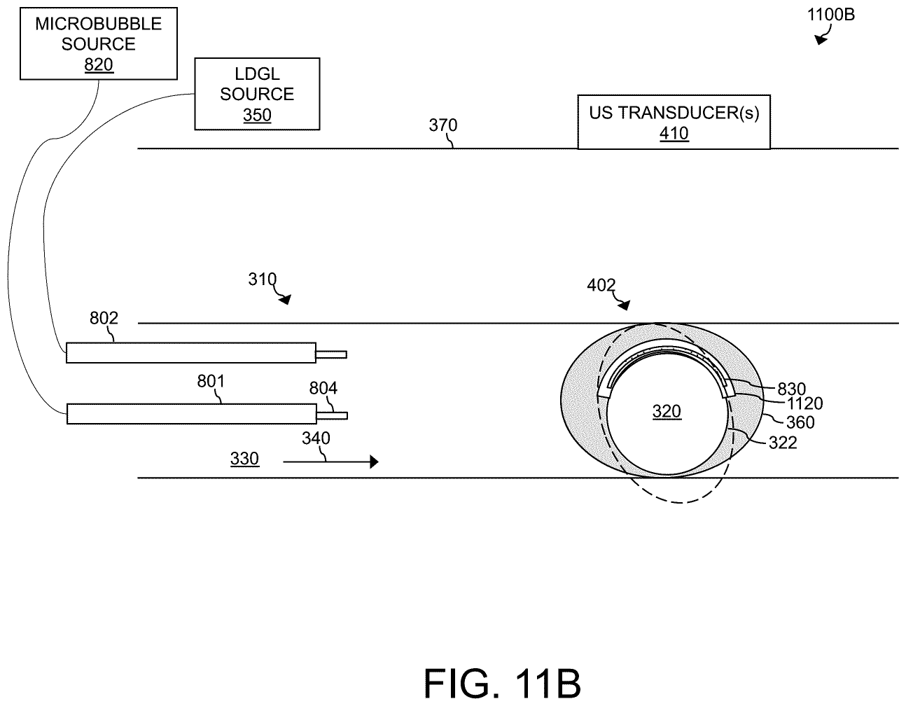

FIG. 11A is a simplified view of an anatomical environment 1100A to illustrate step 705 according to the first embodiment. In environment 1100A, the LDGL 360 has flowed in direction 340 toward the target kidney stone 320 and into the focal zone 400 of the therapeutic ultrasound transducer(s) 410. The LDGL 360 has displaced some or all of the bodily fluids 330 in the focal zone 400, including in the portion 402 of the focal zone 400 between the target kidney stone 320 and the therapeutic ultrasound transducer(s) 410, to thereby reduce the dissolved-gas concentration in the focal zone 400.

The accumulated microbubbles 830 remain on the surface 322 of the target kidney stone 320. In some embodiments, the accumulated microbubbles 830 are in a boundary layer 1120 of bodily fluids 330 along the surface 322 of the target kidney stone 320.

FIG. 11B is a simplified view of an anatomical environment 1100B to illustrate step 705 according to the second embodiment. With respect to step 705, anatomical environments 1100A and 1100B are the same.

In step 706, focused ultrasound energy is applied to the target biomineralization using one or more ultrasound transducers. Step 706 can be the same as step 204.

Since the LDGL reduces cavitation-bubble formation, the focused ultrasound can be applied at a higher peak negative pressure and/or at a lower frequency compared to when the LDGL is not introduced.

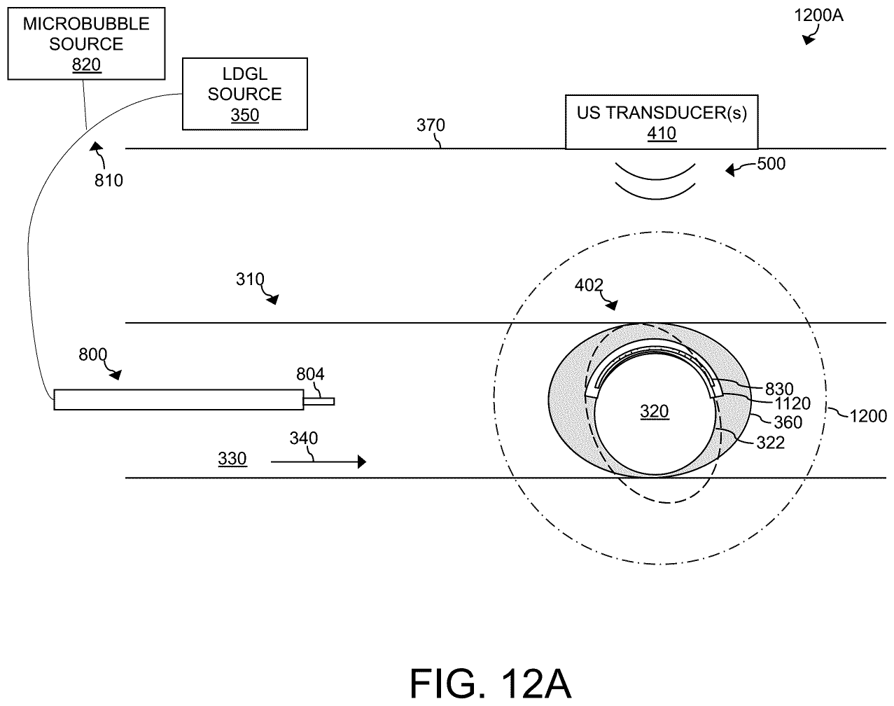

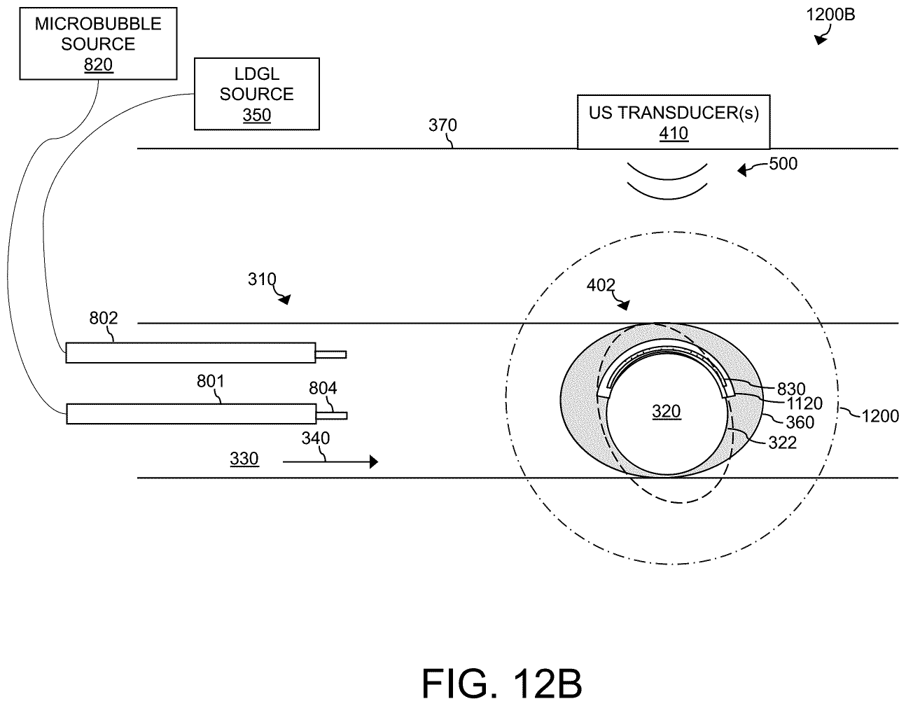

FIG. 12A is a simplified view of an anatomical environment 1200A to illustrate step 706 according to the first embodiment. Anatomical environments 1100A and 1200A are the same except that in anatomical environment 1200A, the therapeutic ultrasound transducer(s) 410 produces focused ultrasound energy 500 that forms and/or defines the focal zone 400.

FIG. 12B is a simplified view of an anatomical environment 1200B to illustrate step 706 according to the second embodiment. With respect to step 706, anatomical environments 1200A and 1200B are the same.

In step 707, the focused ultrasound energy causes the microbubbles to cavitate, which releases energy that fragments the target biomineralization. Gas-bubble shielding can be reduced in the LDGL such that the focused ultrasound energy can pass through the LDGL with reduced and/or minimal reflection, scatter, and/or absorption by gas bubbles. These gas bubbles are formed when the focused ultrasound energy passes through a bodily fluid but are not present (or have a lower concentration) in the LDGL. Additionally or alternatively, cavitation bubble formation can be reduced in the LDGL to reduce the undesirable bioeffects of high-energy cavitation to the surrounding anatomy. In contrast, cavitation bubble formation is promoted at the surface of the target biomineralization by the accumulated microbubbles.

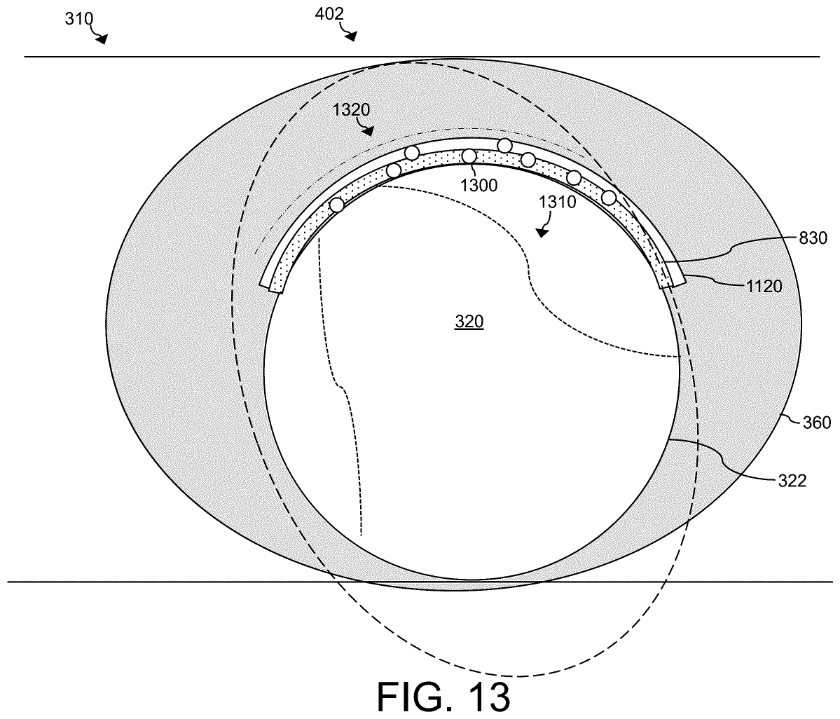

FIG. 13 is an enlarged view of region 1200 in FIGS. 12A and 12B to illustrate step 707 according to the first and second embodiments. The focused ultrasound energy causes the accumulated microbubbles 830 to form cavitation bubbles 1300 in the portion 402 of the focal zone 400 between the target kidney stone 320 and the therapeutic ultrasound transducer(s) 410. The focused ultrasound energy can also cause dissolved gas in the boundary layer 1120 of bodily fluids 330 to form cavitation bubbles 1300. When the cavitation bubbles 1300 collapse (e.g., with positive acoustic pressures), energy is released to fragment 1310 the target kidney stone 320.

It is noted that although the boundary layer 1120, the accumulated microbubbles 830, and the cavitation bubbles 1300 may appear as layers and/or stacked in FIG. 13, the boundary layer 1120, the accumulated microbubbles 830, and the cavitation bubbles 1300 are overlapping in space.

The LDGL reduces gas-bubble shielding and/or spontaneous cavitation bubble formation in a region 1320 that begins at least 2 mm from the surface 322 of the target kidney stone 320. The region 620 can begin at 2 mm from the surface 322, at 3 mm from the surface 322, at 4 mm from the surface, and any other value or range between any two of the foregoing values. The region 1320 is within the portion 402 of the focal zone 400.

In some embodiments, steps 702-707 can be repeated in multiple treatment cycles. The treatment cycles can be repeated immediately or after a delay period.

Though the foregoing examples of methods 20, 70 have been described with respect to kidney stones, it is noted that method 20 and/or method 70 can be applied to other targets such as urinary stones, blood clots (e.g., in coronary arteries), or other targets.

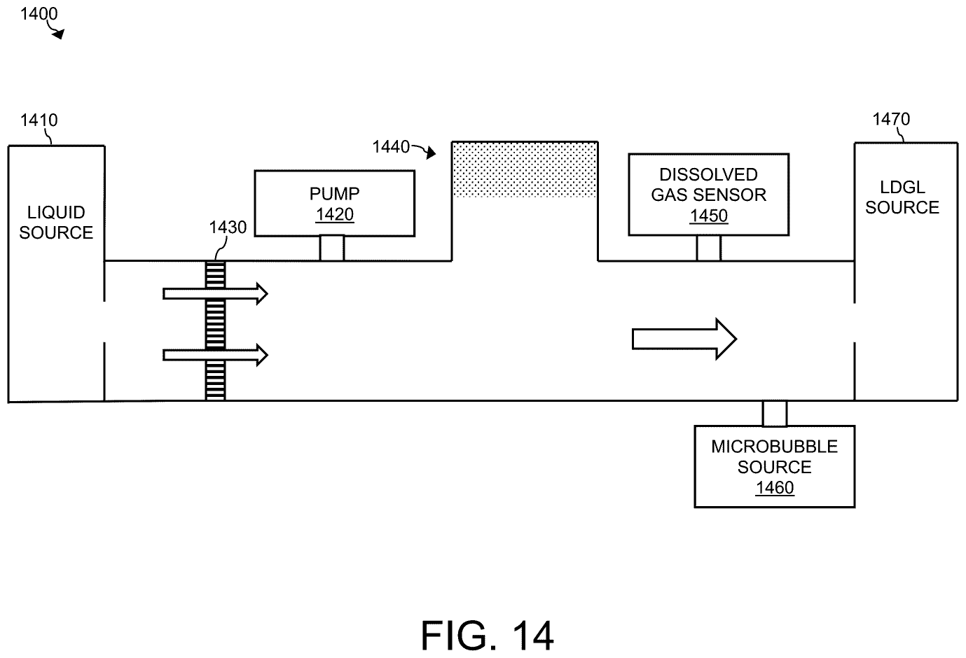

FIG. 14 is a block diagram of an apparatus 1400 for manufacturing an LDGL according to an embodiment. The apparatus 1400 includes a liquid source 1410, a pump 1420, a membrane 1430, a gas trap 1440, a dissolved-gas sensor 1450, an optional microbubble source 1460, and an optional LDGL storage container 1470.

The liquid source 1410 holds a liquid to be used as the LDGF after processing in the apparatus 1400. The liquid can comprise or consist of water, saline, saline, and/or another liquid. The liquid can be biocompatible and suitable to introduce into a mammalian subject, such as a human. For example, the liquid can be or can comprise a sterilized liquid, such as sterile water for injection.

The pump 1420 (e.g., piston pump) causes the liquid in the liquid source 1410 to flow through the membrane 1430. The flow through the membrane 1430 reduces pressure in the liquid, similar to the Venturi effect, and creates turbulent flow causing nucleation, growth, and coalescence of microbubbles. At least some of the dissolved gas in the liquid is diffused into microbubbles that is released into the gas trap 1440. The pump 1420 or another pump can remove the gas from the gas trap 1440 to prevent the gas from dissolving back into the liquid. The pump 1420 can be positioned after (e.g., downstream of) the membrane 1430 to increase the suction and the pressure drop behind the membrane 1430. Alternatively, the pump 1420 can be positioned before (e.g., upstream of) the membrane 1430. The resulting degassed liquid, now an LDGL, flows laterally where its dissolved-gas concentration is measured by the dissolved-gas sensor 1450.

After the dissolved-gas concentration is measured by the dissolved-gas sensor 1450, the optional microbubble source 1460 can add microbubbles to the LDGL. The LDGL with or without the optional microbubbles can then flow through a fluid line, which can be coupled to the optional LDGL storage container 1470.

If the measured dissolved-gas concentration of the LDGL is greater than a maximum or target dissolved-gas concentration, the LDGL can be processed again through the apparatus 1400 to further reduce the dissolved-gas concentration.

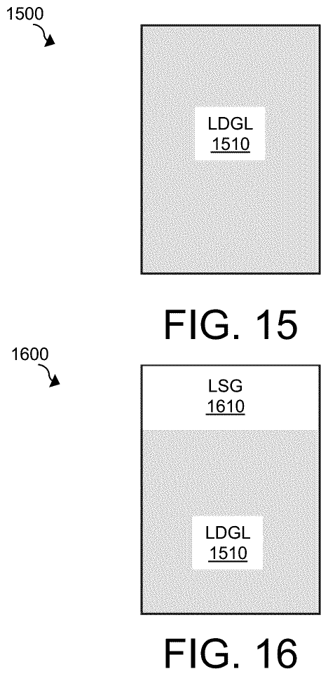

FIGS. 15 and 16 are block diagrams of example LDGL storage containers 1500, 1600, respectively. LDGL storage container 1500 is a gas-tight container that is completely or substantially completely (e.g., 99% by volume) filled with LDGL 1510 such that there is a minimal head space for gas in the LDGL storage container 1500. The minimal head space reduces and/or eliminates the potential for gas to dissolve into the LDGL 1510 during storage.

LDGL storage container 1600 is a gas-tight container that is partially filled with LDGL 1510 and the remaining volume is filled with a low-solubility gas 1610. The low-solubility gas (LSG) 1610 that contacts the LDGL 1510 but only minimally dissolves into the LDGL 1510, thereby reducing and/or eliminating the potential for gas to dissolve into the LDGL 1510 during storage. Examples of LSG 1610 include perfluoronated hydrocarbons.

The LDGL storage containers 1500, 1600 can be made of glass, metal, or another gas-impermeable material. LDGL 1510 can be the same as LDGL 360.

The invention should not be considered limited to the particular embodiments described above, but rather should be understood to cover all aspects of the invention as fairly set out in the attached claims. Various modifications, equivalent processes, as well as numerous structures to which the invention may be applicable, will be apparent to those skilled in the art to which the invention is directed upon review of this disclosure. The claims are intended to cover such modifications and equivalents.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

What is claimed is:

1. A method for treating a target biomineralization in a mammalian subject, comprising:
placing a catheter in proximity to the target biomineralization, the catheter fluidly coupled to a source of a low-dissolved-gas liquid (LDGL), the LDGL having a dissolved-gas concentration of less than or equal to about 20% of an oxygen saturation level of the LDGL;
introducing the LDGL through the catheter;
flowing the LDGL towards the target biomineralization such that at least some of the LDGL is in a focal zone of one or more ultrasound transducers and between the target biomineralization and the one or more ultrasound transducers, the focal zone aligned with the target biomineralization;
applying focused ultrasound energy to the target biomineralization with the one or more ultrasound transducers, the focused ultrasound energy passing through the LDGL; and
forming cavitation bubbles, with the focused ultrasound energy, at a surface of the target biomineralization to fragment the target biomineralization,
wherein the LDGL reduces acoustic shielding in the focal zone between the target biomineralization and the one or more ultrasound transducers compared to when the LDGL is not introduced.

2. The method of claim 1, wherein the catheter is placed upstream of the target biomineralization such that bodily fluids flow from the catheter towards the target biomineralization.

3. The method of claim 1, wherein the LDGL reduces the acoustic shielding in a region that begins at least 2 mm from the surface of the target biomineralization.

4. The method of claim 3, wherein the LDGL reduces spontaneous cavitation in the region compared to when the LDGL is not introduced.

5. The method of claim 1, further comprising introducing a stream of the LDGL through the catheter.

6. The method of claim 1, wherein:
when the at least some of the LDGL is in the ultrasound focal zone and between the target biomineralization and the one or more ultrasound transducers, a boundary layer of bodily fluids is located along the surface of the target biomineralization, and
the cavitation bubbles are formed in the boundary layer of bodily fluids.

7. The method of claim 1, wherein a dissolved-oxygen concentration of the LDGL less than or equal to about 2 mg/L.

8. A method for treating a target biomineralization in a mammalian subject, comprising:
placing a catheter in proximity to the target biomineralization, the catheter fluidly coupled to a source of a low-dissolved-gas liquid (LDGL) and to a source of microbubbles, the LDGL having a dissolved-gas concentration of less than or equal to about 20% of an oxygen saturation level of the LDGL;
introducing the microbubbles through the catheter;
flowing the microbubbles towards the target biomineralization such that at least some of the microbubbles accumulate on a surface of the target biomineralization, the at least some of the microbubbles in a focal zone of one or more ultrasound transducers and between the target biomineralization and the one or more ultrasound transducers, the focal zone aligned with the target biomineralization;
introducing the LDGL through the catheter;
flowing the LDGL towards the target biomineralization such that at least some of the LDGL is in the focal zone of the one or more ultrasound transducers and between the target biomineralization and the one or more ultrasound transducers;
applying focused ultrasound energy to the target biomineralization with the one or more ultrasound transducers, the focused ultrasound energy passing through the LDGL; and
forming cavitation bubbles with the accumulated microbubbles on the surface of the target biomineralization to fragment the target biomineralization, the cavitation bubbles formed with the focused ultrasound energy,
wherein the LDGL reduces an acoustic shielding in the focal zone between the target biomineralization and the one or more ultrasound transducers compared to when the LDGL is not introduced.

9. The method of claim 8, wherein the catheter is placed upstream of the target biomineralization such that bodily fluids flow from the catheter towards the target biomineralization.

10. The method of claim 8, wherein the LDGL reduces the acoustic shielding in a region that begins at least 2 mm from the surface of the target biomineralization.

11. The method of claim 10, wherein the LDGL reduces spontaneous cavitation in the region compared to when the LDGL is not introduced.

12. The method of claim 8, further comprising introducing a stream of the LDGL through the catheter.

13. The method of claim 8, wherein:
when the at least some of the LDGL is in the focal zone and between the target biomineralization and the one or more ultrasound transducers, a boundary layer of bodily fluids is located along the surface of the target biomineralization, and
the accumulated microbubbles are in the boundary layer of bodily fluids.

14. The method of claim 8, wherein a Y-coupling fluidly couples the catheter to the source of the LDGL and to the source of microbubbles.

15. A method for treating a target biomineralization in a mammalian subject, comprising:
placing a first catheter in proximity to the target biomineralization, the first catheter fluidly coupled to a source of microbubbles;
introducing the microbubbles through the first catheter;
flowing the microbubbles towards the target biomineralization such that at least some of the microbubbles accumulate on a surface of the target biomineralization, the at least some of the microbubbles in a focal zone of one or more ultrasound transducers and between the target biomineralization and the one or more ultrasound transducers, the focal zone aligned with the target biomineralization;
placing a second catheter in proximity to the target biomineralization, the second catheter fluidly coupled to a source of a low-dissolved-gas liquid (LDGL), the LDGL having a dissolved-gas concentration of less than or equal to about 20% of an oxygen saturation level of the LDGL;
introducing the LDGL through the second catheter;
flowing the LDGL towards the target biomineralization such that at least some of the LDGL is in the focal zone of the one or more ultrasound transducers and between the target biomineralization and the one or more ultrasound transducers;
applying focused ultrasound energy to the target biomineralization with the one or more ultrasound transducers, the focused ultrasound energy passing through the LDGL; and
forming cavitation bubbles with the accumulated microbubbles on the surface of the target biomineralization to fragment the target biomineralization, the cavitation bubbles formed with the focused ultrasound energy,
wherein the LDGL reduces an acoustic shielding in the focal zone between the target biomineralization and the one or more ultrasound transducers compared to when the LDGL is not introduced.

16. The method of claim 15, wherein the first and/or second catheters are placed upstream of the target biomineralization such that bodily fluids flow from the first and second catheters towards the target biomineralization.

17. The method of claim 15, wherein the LDGL reduces the acoustic shielding in a region that begins at least 2 mm from the surface of the target biomineralization.

18. The method of claim 17, wherein the LDGL reduces spontaneous cavitation in the region compared to when the LDGL is not introduced.

19. The method of claim 15, further comprising introducing a stream of the LDGL through the catheter.

20. The method of claim 15, wherein:
when the at least some of the LDGL is in the focal zone and between the target biomineralization and the one or more ultrasound transducers, a boundary layer of bodily fluids is located along the surface of the target biomineralization, and
the accumulated microbubbles are in the boundary layer of bodily fluids.
